(19) United States
(12) Patent Application Publication
(10) Pub. No.: US 2022/0031629 A1
(43) Pub. Date: Feb. 3, 2022

(54) OPHTHALMIC COMPOSITIONS FOR REMOVING MEIBUM OR INHIBITING MEIBUM BUILDUP

(71) Applicant: Altaire Pharmaceuticals, Inc., Aquebogue, NY (US)

(72) Inventor: Assad Sawaya, Baiting Hollow, NY (US)

(21) Appl. No.: 17/389,680

(22) Filed: Jul. 30, 2021

Related U.S. Application Data
(60) Provisional application No. 63/059,275, filed on Jul. 31, 2020.

(51) Int. Cl. A61K 9/51; A61K 9/00

(52) U.S. Cl. CPC A61K 9/5146; B82Y 5/00; A61K 9/0048

(57) ABSTRACT

Microemulsion formulations containing polyoxyethylene castor oil derivatives (such as polyoxyl 35 castor oil) contain nanoparticle micelles and are capable of dissolving/absorbing meibum materials. By removing obstructive meibum materials that may be positioned in the orifice of meibomian gland channels, the present formulations provide a non-irritating treatment for meibomian gland dysfunction (MGD).

### CROSS-REFERENCE TO RELATED APPLICATIONS

This application claims the benefit of, and priority to, U.S. Provisional Patent Application No. 63/059,275, filed on Jul. 31, 2020, the entire contents of which are hereby incorporated herein by reference.

### TECHNICAL FIELD

This disclosure relates to artificial tear compositions useful, for example, in treating dry eye disease and meibomian gland dysfunction (MGD). In particular, this disclosure relates to ophthalmic formulations that cleanse they eye of a patient by removing or inhibiting buildup of meibum, for example in the channels of the meibomian glands.

### BACKGROUND

Meibomian Gland Dysfunction (MGD) is an eye disease which may cause dry eyes. The meibomian glands are a group of sebaceous glands located on the inside of the eyelids which produce oily lipid substances or meibum, inhibiting the tear film from evaporation. MGD may be associated the obstruction of the meibomian gland channels by a buildup of meibum. Chemically, human meibum is composed primarily of wax esters, cholesteryl esters, triglycerides, free fatty acid, free sterols, alcohols, diglycerides, monoglycerides, phospholipids, and proteins.

Approaches for MGD treatment include physical compression, eye drops, nutrition, and the use of drugs. Physical compression methods include intra-ductal meibomian gland probing and LIPIFLOW technology. Intra-ductal meibomian gland probing is a method to relieve MGD symptoms by physically penetrating the blocked meibomian gland with a metal probe. LIPIFLOW technology applies heat to the upper and lower eyelids while simultaneously pressing the extremal eyelid to force out the blocking meibum. Nutrition methods include the intake of omega 3-fatty acids and their derivatives (esters) to influence the metabolism and to improve the quality of expressed meibum. Drugs used to treat MGD include anti-inflammatory/antibiotic agents, such as cyclosporine and azithromycin.

Another treatment for MGD is the use of eye drops. For example, lubricant artificial tears containing celluloses and hyaluronic acid help in supplementing the aqueous layer. Certain white, milky emulsion eye drop products which contain oily lipids are capable of replenishing, stabilizing, and thus controlling the integrity of the tear film. However, artificial tears and emollient eye drops currently available are limited in terms of their ability to rinse and cleanse the eyes to treat MGD in that they are either easily flushed away, exhibit poor affinity towards meibum materials, or impart cloudiness and cause blurring of the patient's vision.

It would be desirable to develop eye drop products that remove meibum obstructions and/or inhibit obstruction of the meibomian gland channels by a buildup of meibum as a treatment for MGD or other dry eye conditions.

### SUMMARY

Ophthalmic formulations in accordance with the present disclosure include nanoparticle micelles formed by polyoxyethylene castor oil derivatives, such as polyoxyl 35 castor oil, and can dissolve meibum and encapsulate meibum components, such as, for example, cholesteryl esters and wax esters, for ultimate removal from the eye. In this manner, formulations in accordance with the present disclosure remove meibum obstructions and/or inhibit obstruction of the meibomian gland channels by a buildup of meibum, providing a treatment for MGD or other dry eye conditions. The formulations are stable and may have a particle size less than about 100 nm, in embodiments from about 10 nm to about 20 nm prior to instillation in the eye of a patient, providing a clear solution appearance.

The use of topical cleansing eye drop compositions in accordance with the present disclosure as a cleansing treatment for MGD has the advantage of user-friendliness, lower expense, and increased efficiency.

While the following discussion focusses on cholesteryl/sterol esters and wax esters, it should be understood that other components of meibum may be dissolved and encapsulated by the nanoparticles in the compositions in accordance with the present disclosure.

As used herein, the terms “a” or “an” means that “at least one” or “one or more” unless the context clearly indicates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments.

As used herein, the terms “treat,” “treated,” or “treating” mean both therapeutic treatment and prophylactic or preventative measures wherein the object is to prevent or slow down (lessen) an undesired physiological condition, disorder or disease, or obtain beneficial or desired clinical results. Beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response without excessive levels of side effects.

### BRIEF DESCRIPTION OF THE DRAWINGS

A more complete understanding of the presently disclosed concepts and illustrative embodiments may be acquired by referring to the following description, taken in conjunction with the figures of the accompanying drawings wherein:

FIG. 1 shows a plot of the mean particle size as a function of the mixing time in hours when 3 gm of castor oil was added at 37° C. into 100 mL of a composition in accordance with the present disclosure containing 40% polyoxyl 35 castor oil;

FIG. 2 shows the interaction of individual meibum-like material with a cleansing composition in accordance with the present disclosure containing 40% polyoxyl 35 castor oil (mixing at 37° C. at 135 RPM);

FIG. 3 shows a plot of particle size as a function of polyoxyl 35 castor oil concentration when 0.5 gm of castor oil was added into 100 ml of cleansing formulations in accordance with the present disclosure containing 5%-40% polyoxyl 35 castor oil under the conditions of mixing at 700 RPM and at temperature 37° C. for 18 hours individually (except for the formulation containing 40% polyoxyl 35 castor oil for which the mixing time was 3 hours and mixing speed was 350 RPM);

FIG. 4 shows the particle size distribution (Gaussian) of a cleansing formula with 8% polyoxyl 35 castor oil before (left) and after (right) the interaction with meibum; and

FIG. 5 shows the meibum dissolved/absorbed in Percentage (%) as a function of the concentration of polyoxyl 35 castor oil in the cleansing formulation.

### DETAILED DESCRIPTION

In illustrative embodiments described herein, clear ophthalmic compositions include nanoparticles of polyoxyethylene castor oil derivatives capable of dissolving meibum and encapsulating meibum components, such as, for example, cholesteryl/sterol esters and wax esters.

Polyoxyethylene castor oil derivatives are, essentially, the nanolipid or nanoparticle source in the presently described cleansing formulations. Polyoxyethylene castor oil derivatives contain both a) hydrophilic parts and b) hydrophobic parts. In aqueous solution, polyoxyethylene castor oil derivative molecules self-assemble with each other to form micelles (nanolipids), which are stable nanoparticles. The structure of those micelles is believed to have a hydrophobic “core” and a hydrophilic “shell”.

Polyoxyethylene castor oil derivatives are produced by reacting varying amounts of ethylene oxide with castor oil under raised pressure and temperature in the presence of a catalyst. Different polyethoxylated castor oils can be produced by controlling the molar ratio of ethylene oxide relative to castor oil. Polyoxyethylene castor oil derivatives (sometimes also called polyoxyl castor oil, polyoxyl n castor oil, polyethylene glycol castor oil, castor oil ethoxylates, or polyethoxylated castor oil) are complex mixtures of various hydrophobic and hydrophilic components. In the polyethoxylated castor oil, the hydrophobic constituents make up about 80% of the total mixture, the main component being glycerol polyethylene glycol ricinoleate. Other hydrophobic constituents include fatty acid esters of polyethylene glycol along with some unchanged castor oil. The hydrophilic part consists of polyethylene glycols and glycerol ethoxylates.

When referred to as “polyoxyl n castor oil”, the number (n) associated with the name of the substance represents the average number of oxyethylene units in the compound. Polyoxyl n castor oil where n=30 to 40 is a mixture of tri-ricinoleate esters of ethoxylated glycerol with small amounts of polyethyleneglycol (macrogol) ricinoleate and the corresponding free glycols. Polyoxyl n hydrogenated castor oil where n=40 to 60 is a mixture of tri-hydroxystearate esters of ethoxylated glycerol with small amounts of macrogol tri-hydroxystearate and the corresponding free glycols. Polyoxyl castor oils and polyoxyl hydrogenated castor oils are nonionic surfactants.

Suitable polyoxyethylene castor oil derivatives include polyoxyl 35 castor oil. KOLLIPHOR EL, formerly known as CREMOPHOR EL, is the registered trademark of BASF Corp. for its version of polyethoxylated castor oil. It is prepared by reacting 35 moles of ethylene oxide with each mole of castor oil. The resulting product is a mixture (CAS number 61791-12-6): the major component is the material in which the hydroxyl groups of the castor oil triglyceride have ethoxylated with ethylene oxide to form polyethylene glycol ethers. Minor components are the polyethylene glycol esters of ricinoleic acid, polyethylene glycols and polyethylene glycol ethers of glycerol. Polyoxyl 35 castor oil is also commercially available from Croda under the trade name of ETOCAS 35.

Other examples of suitable polyoxyethylene castor oil derivatives include polyoxyl 40 castor oil (commercially available from Sasol Performance Chemicals under the trade name MARLOWET 40, and commercially available from BASF under the trade name EMULGIN RO 40), polyoxyl 40 hydrogenated castor oil (commercially available from BASF under the trade name CREMOPHOR RH 40) and polyoxyl 60 hydrogenated castor oil (commercially available from BASF under the trade name CREMOPHOR RH 60).

The concentration of polyoxyethylene castor oil derivative in the present compositions may be sufficient to encapsulate non-polar lipids, such as wax esters and cholesteryl esters, while maintaining a clear solution appearance. In embodiments, the polyoxyethylene castor oil derivative(s) is present in the formulation in an amount from about 2% to about 70% by weight; in embodiments, from about 1% to about 50% by weight. Where the polyoxyethylene castor oil derivative employed is polyoxyl 35 castor oil, the concentration of polyoxyl 35 castor oil in the present compositions may be sufficient to encapsulate non-polar lipids, such as wax esters and cholesteryl esters, while maintaining a clear solution appearance. In embodiments, polyoxyl 35 castor oil is present in the formulation in an amount from about 5% to about 50% by weight; in embodiments, from about 8% to about 40% by weight; in yet other embodiments from about 18% to about 35% by weight.

As previously mentioned, the polyoxyethylene castor oil derivatives form nanoparticles (micelles) that make up the present clear ophthalmic compositions. The formulations are capable of dissolving meibum and the micelles encapsulate meibum components, such as, for example, cholesteryl/sterol esters and wax esters.

Without wishing to be bound by any theory, it is believed that increasing the concentration of the polyoxyethylene castor oil derivatives, and hence increasing the population of nanolipids/nanoparticles, provides a greater number of polarity “vacancies” leading to a greater amount of the meibum materials being absorbed. In embodiments, formulations in accordance with the present disclosure contain a sufficient amount of polyoxyethylene castor oil derivative(s) to produce 2.5×10¹⁷ nanoparticles per milliliter of solution. The calculated number of nanoparticles per ml for certain illustrative compositions containing varying amounts of polyoxyl 35 castor oil as the polyoxyethylene castor oil derivative is presented in Table A.

TABLE A

| Amount of PEG 35 | Mean Particle | Number of |
|---|---|---|
| Castor Oil | Size* | Nanoparticles per ml** |
| 1% | 11.9 nm | 1.4 × 10¹⁶ |
| 3% | 12.9 nm | 3.4 × 10¹⁶ |
| 5% | 12.9 nm | 5.6 × 10¹⁶ |
| 8% | 10.8 nm | 1.5 × 10¹⁷ |
| 10% | 10.6 nm | 2.0 × 10¹⁷ |
| 15% | 11.5 nm | 2.4 × 10¹⁷ |
| 20% | 12.2 nm | 2.7 × 10¹⁷ |
| 25% | 11.3 nm | 4.2 × 10¹⁷ |
| 30% | 10.7 nm | 5.9 × 10¹⁷ |
| 35% | 11.7 nm | 5.3 × 10¹⁷ |
| 40% | 12.2 nm | 5.3 × 10¹⁷ |

*tested value using a DLS particle sizer, (PSS, Santa Barbara, CA)
**calculated value based on spherical particle shape of the polyoxyl 35 castor oil nanoparticles with known concentration.

In embodiments, the compositions are substantially free of nonpolar lipid compounds (e.g., triglycerides such as, by way of non-limiting example, castor oil, etc.) to ensure that the hydrophobic “core” of the polyoxyethylene castor oil micelles (nanolipids) are substantially “empty” and present a large number of polarity “vacancies” that are available to help absorb meibum materials. In embodiments, the present compositions contain less than 0.01% added non-polar lipids. In embodiments, the present compositions contain less than 0.001% added non-polar lipids. In embodiments, the present compositions contain no added non-polar lipids. By the term “added” it is meant that the non-polar lipid is a separate ingredient, because, as those skilled in the art will appreciate, some components of the compositions may include a small amount of non-polar lipids (e.g., the polyoxyethylene castor oil derivatives may contain a small amount of castor oil remaining from the manufacture of the ingredient).

In embodiments, the compositions are substantially free of polar lipid compounds (e.g., phospholipids such as, by way of non-limiting example, soy lecithin and 1,2-dimyristoyl-sn-glycero-3-phosphoglycerol, sodium salt, etc.) to ensure that the hydrophobic “core” of the polyoxyethylene castor oil micelles (nanolipids) are substantially “empty” and present a large number of polarity “vacancies” that are available to help absorb meibum materials. In embodiments, the present compositions contain less than 0.01% added polar lipids. In embodiments, the present compositions contain less than 0.001% added polar lipids. In embodiments, the present compositions contain no added polar lipids. By the term “added” it is meant that the polar lipid is a separate ingredient, because, as those skilled in the art will appreciate, some components of the compositions may include a small amount of polar lipids.

In embodiments, one or more buffering agents may be included in the present compositions. Any buffering agent known to be suitable for ophthalmic compositions may be used. In embodiments, the buffering agents may include one or more of boric acid, sodium borate, sodium hydroxide, tromethamine, amino methyl propanol, or combinations thereof. Suitable combinations of buffering agents useful in the present compositions include: a) boric acid and sodium borate, b) boric acid and sodium hydroxide, c) boric acid and tromethamine, d) boric acid and amino methyl propanol, or combinations thereof. In embodiments, the buffering agents employed are boric acid and tromethamine.

The total concentration of buffering agents used in the present compositions may be up to 5%, depending on the buffering agent or combination of buffering agents chosen. In embodiments, the buffering agent includes boric acid in an amount from about 0.1% to about 2% by weight; in embodiments, from about 0.5% to about 1%. In embodiments, the buffering agent includes tromethamine, alone or in combination with boric acid. When present, the amount of tromethamine in the present composition may be from about 0.05% to about 1% by weight; in embodiments, tromethamine is present in an amount from about 0.1% to about 0.5%.

In embodiments, one or more chelating agents may be included in the present compositions. Any chelating agent known to be suitable for ophthalmic compositions may be used. A non-limiting example of a suitable chelating agent is edetate disodium (EDTA). The total concentration of chelating agents used in the present compositions may be up to 2%. In embodiments where EDTA is used as the chelating agent, EDTA may be present in the composition in an amount from about 0.01% to about 0.1% by weight.

In embodiments, one or more lubricant/demulcent agents may be included in the present compositions. Any lubricant/demulcent agent known to be suitable for ophthalmic compositions may be used. Non-limiting examples of suitable lubricant/demulcent agents include glycerin, propylene glycol, sorbitol, polyethylene glycol 400 (PEG 400), and polysorbate 80. It should, of course, be understood that combinations of these lubricant/demulcent agents may be included in the present compositions. The total concentration of lubricant/demulcent agents used in the present compositions may be up to 5%. In embodiments where glycerin is used as the lubricant/demulcent agent alone or in combinations with other lubricant/demulcent agents, glycerin may be present in the present compositions in an amount from about 0.1% to about 1% by weight. In embodiments where propylene glycol is used as the lubricant/demulcent agent alone or in combinations with other lubricant/demulcent agents, propylene glycol may be present in the present compositions in an amount from about 0.1% to about 0.5% by weight. In embodiments where sorbitol is used as the lubricant/demulcent agent alone or in combinations with other lubricant/demulcent agents, sorbitol may be present in the present compositions in an amount from about 0.1% to about 0.5% by weight. In embodiments where PEG 400 is used as the lubricant/demulcent agent alone or in combinations with other lubricant/demulcent agents, PEG 400 may be present in the present compositions in an amount from about 0.1% to about 0.5% by weight. In embodiments where polysorbate 80 is used as the lubricant/demulcent agent alone or in combinations with other lubricant/demulcent agents, polysorbate 80 may be present in the present compositions in an amount from about 0.1% to about 1.0% by weight.

In embodiments, one or more gelling agents may be included in the present compositions. Any gelling agent known to be suitable for ophthalmic compositions may be used. Non-limiting examples of suitable gelling agents include, for example, ionic and nonionic polysaccharides, such as, for example, guar gum, gellan gum, xantham gum, and the like. In embodiments, gellan gum is used as the gelling agent. Gellan gum is commercially available, for example, from Kelco under the trade name GELRITE. GELRITE gellan gum interestingly starts gelling when exposed to metal ions present in the tear film. The total concentration of gelling agents used in the present compositions may be up to 2%. In embodiments where gellan gum is used as the gelling agent alone or in combinations with other gelling agents, gellan gum may be present in the present compositions in an amount from about 0.001% to about 0.5% by weight; in embodiments, from about 0.002% to about 0.1%.

In embodiments, one or more thickening agents may be included in the present compositions. Any thickening agent known to be suitable for ophthalmic compositions may be used. Non-limiting examples of suitable thickening agents include, for example, hypromellose or hydroxypropyl methylcellulose (HPMC), hydroxyethylcellulose (HEC), carboxymethylcellulose sodium (CMC Na), polyvinyl alcohol (PVA), povidone, and sodium hyaluronate. In embodiments, HEC is used as the thickening agent. HEC is commercially available, for example, from Hercules under the trade name NATROSOL. Among the useful HEC products available from Hercules is NATROSOL 250M. The total concentration of thickening agents used in the present compositions may be up to 2%. In embodiments where HEC is used as the thickening agent alone or in combinations with other thickening agents, HEC may be present in the present compositions in an amount from about 0.1% to about 0.5% by weight.

In embodiments, a preservative system may be included in the present compositions. Any preservative system known to be suitable for ophthalmic compositions may be used. Non-limiting examples of suitable preservatives include, for example, benzalkonium chloride, stabilized chlorine peroxide complexes, stabilized oxychloro complexes, polyquaternary ammonium compounds such as polyquaternium-1 (POLYQUAD), polyquaternium-42 (BUSAN 1507, Buckman Laboratories), and polyhexamethylene biguanide (PHMB or polyhexanide), chlorobutanol, thimerosal, sorbic acid, and chlorhexidine gluconate. Stabilized Oxychloro Complex (SOC) is an oxidative (antimicrobial) preservative containing a mixture of oxychloro species, predominantly chlorite (at 99.5%), chlorate (at 0.5%) and traces of chlorine dioxide. SOC is used in eye drops as a gentle preservative that converts to sodium and chloride ions, oxygen and water on the ocular surface and is well tolerated and leaves no preservative residue in the eye. In embodiments, SOC preservatives used in the present compositions may be present in an amount up to 200 ppm. In embodiments where SOC is used as the preservative alone or in combinations with other preservatives, SOC may be used in the present compositions in an amount from about 50 ppm to about 100 ppm. In embodiments, polyhexamethylene biguanide (PHMB or polyhexanide) is used as the preservative. The total concentration of preservative PHMB used in the present compositions may be up to 10 ppm. In embodiments where PHMB is used as the preservative alone or in combinations with other preservatives, PHMB may be used in the present compositions in an amount from about 1 ppm to about 4 ppm.

Microemulsion systems in accordance with the present disclosure can be further utilized to carry pharmaceutically active agents in the nanoparticle capsules, including glaucoma therapeutics, pain relievers, anti-inflammatory compounds, anti-allergy medications, and anti-microbials. Non-limiting examples of pharmaceutically active agents that may be included in the present ophthalmic compositions include Naphazoline HCl, Tetrahydrozoline HCl, Cyclosporine, Timolol, Dorzolamide, Pilocarpine, Brimonidine Tartrate, Olopatadine, Epinastine, Betaxolol, Pheniramine Maleate, Lotepredenol Etabonate, Ciprofloxacin, Ofloxacin, Gentamicin, Flurbiprofen, Gramicidin, Erythromycin, Levofloxacin, Moxifloxacin, Neomycin, Polymyxin B, Sodium Sulfacetamide, Tobramycin, Bacitracin, Dexamethasone, Flurometholone, Hydrocortisone, Prednisolone, Levalbuterol Hydrochloride, Trifluridine, Naproxen, Diclofenac, Bromfenac, Ketotifen Fumarate, Travoprost, Latanoprost, Bimatoprost, Tropicamide, Phenylephrine, Tetracaine, Proparacaine, Benoxinate, and Lidocaine. In embodiments, the clear microemulsion compositions in accordance with the present disclosure contain sufficient hydrophobic groups to serve as a vehicle to solubilize/deliver some poorly water-soluble pharmaceutically active drugs, e.g. cyclosporine, erythromycin, hydrocortisone, bacitracin, prostaglandins, etc.

The pharmaceutically active agent(s) may be incorporated into the present compositions in a therapeutically effective amount. The term “effective amount” or “therapeutically effective amount” is an amount sufficient to affect a therapeutically beneficial or therapeutically desired result. A therapeutically effective amount can be administered in one or more administrations, applications or dosages. In embodiments, pharmaceutically active agent(s) may be incorporated into the present compositions in an amount from 0.01% to 1.0% by weight.

In embodiments, the present compositions may also be prepared as preservative-free compositions. The compositions, whether prepared with or without any preservative, will be sterilized as part of the manufacturing process. In some embodiments, the composition may be sterilized by heating the composition to a temperature that eliminates any bacteria or other living microorganisms. In other embodiments, the composition may be sterilized by passing it through a membrane filter having a sufficiently small pore size (e.g., 0.2 μm membrane filter) to eliminate any bacteria or other living microorganisms. In embodiments, the present composition is passed through a 0.2 μm membrane filter to provide a preservative-free composition.

The following Table 1 provides ranges of ingredients for illustrative embodiments of compositions in accordance with this disclosure.

TABLE 1
Cleansing Compositions

| mg/mL | Ingredient | Percentage |
|---|---|---|
| Qs. 1.0 mL | Water for Injection, USP | Qs. 100% |
| 5.00-15.00 | Boric Acid, NF | 0.5%-1.5% |
| 1.00-10.00 | Tromethamine and/or | 0.1%-1% |
| | Aminomethylpropanol | |
| 1.00-10.00 | PEG 400, USP | 0.1%-1% |
| 1.00-10.00 | Propylene Glycol, USP | 0.1%-1% |
| 1.00-10.00 | Sorbitol, 70% Solution, USP | 0.1%-1% |
| 1.00-10.00 | Polysorbate 80 | 0.1%-1% |
| 1.00-10.00 | Glycerine, USP | 0.1%-1% |
| 10.00-500.00 | Polyoxyl 35 Castor Oil, NF | 1%-50% |
| <0.05 | Stabilized Oxychloro Complex | <0.05% |

The present compositions may be prepared using any technique within the purview of those skilled in the art. In embodiments, the compositions are prepared by combining the ingredients, mixing, and heating if needed. Mixing should be continued until a clear, colorless, or slightly yellowish solution forms. For convenience, or compatibility reasons, the components of the formulations may be parsed into two parts, with the first and second parts being prepared separately, and then combined to make the final formulation.

In embodiments, the appearance of the present compositions may be clear to colorless. In other embodiments, the appearance of the present composition may be pale yellow. As a practical matter, body temperature (about 37° C.) is the working temperature for an eye drop product, including the present cleansing formulations, to take its “therapeutic function for cleansing purposes. Accordingly, in embodiments, the appearance of the present compositions may be clear to colorless at 37° C. In embodiments, the appearance of the present composition is neither cloudy nor milky.

In embodiments, the size of nanoparticles in the microemulsion system of the present composition may be from about 2 nm to about 100 nm, in embodiments from about 5 nm to about 50 nm, in other embodiments from about 10 nm to about 25 nm, as observed utilizing a submicron particle sizer (available, e.g., from Particle Sizing Systems, Santa Barbara, Calif. now part of Entegris Inc., Billerica, Mass., USA). It should be understood that the nanoparticle sizes mentioned above are in the formulation prior to instillation into a patient's eye. Once administered to a patient, those skilled in the art reading this disclosure will appreciate that the size of the nanoparticles in the formulation may increase due to the absorption of meibum material. In embodiments, the size of the nanoparticles in the formulation after instillation into a patient's eye and the absorption of meibum material remains below 100 nm, in embodiments, below about 50 nm and the formulation maintains a clear appearance.

In embodiments, the tonicity of an eye drop in accordance with embodiments of the present compositions may be from about 280 mOsm/kg to about 330 mOsm/kg.

In embodiments, the pH of the present composition may be from about 6.0 to about 8.0.

In embodiments, the viscosity of an eye drop in accordance with embodiments of the present compositions may be from about 5 cps to about 50 cps.

In embodiments, the viscosity of an ointment/gel in accordance with embodiments of the present compositions may be from about 100,000 cps to about 800,000 cps.

In embodiments, the specific gravity of the present composition may be from about 1.000 to about 1.020.

### EXAMPLES

The following examples are presented to illustrate specific aspects of compositions in accordance with the present disclosure and their properties. They are different composition systems to reflect various aspects of non-limiting, illustrative examples of nanoparticle microemulsions.

### Example 1

Meibum can be dissolved and meibum components sequestered inside nanoparticles using compositions in accordance with the present disclosure. Once captured within the nanoparticles, the meibum components may be removed from the eye of the patient by flushing the eye. Thus, the present formulations when used as eye drop products are capable of treating a patient suffering from dry eye or MGD caused in part by meibum materials obstructing the orifice of meibomian gland channels.

Compositions containing 5% to 40% polyoxyl 35 castor oil are prepared using the general formulation presented in Table 2.

TABLE 2

| mg/mL | Ingredient | Percentage |
|---|---|---|
| Qs. 1.0 mL | Water for Injection, USP | Qs. 100% |
| 0-11.20 | Boric Acid, NF | 0%-1.12% |
| 0-6.50 | Tromethamine and/or | 0%-0.65% |
| | Aminomethylpropanol | |
| 4.00 | PEG 400, USP | 0.4% |
| 3.00 | Propylene Glycol, USP | 0.3% |
| 5.00 | Sorbitol, 70% Solution, USP | 0.35% |
| 5.00 | Polysorbate 80 | 0.5% |
| 5.00 | Glycerine, USP | 0.5% |
| 50.00-400.00 | Polyoxyl 35 Castor Oil, NF | 5%-40% |
| 2.00 | PHMB, 0.1% Solution | 0.0002% |

The compositions containing 5% to 40% polyoxyl 35 castor oil are clear solutions with particles in the range of 5 nm to 50 nanometers, as summarized in Table 3 below.

TABLE 3
Particle size and viscosity of various Formulations (5%-40%)

| Concentration of | | | |
|---|---|---|---|
| Polyoxyl 35 Castor Oil | Mean Particle Size | Viscosity | |
| 5% | 12.9 nm | 1.3 | cps |
| 8% | 10.8 nm | 1.8 | cps |
| 10% | 10.6 nm | 2.2 | cps |
| 15% | 11.5 nm | 3.8 | cps |
| 20% | 12.2 nm | 6.6 | cps |
| 25% | 11.3 nm | 18 | cps |
| 30% | 10.7 nm | 47 | cps |
| 35% | 11.7 nm | 155 | cps |
| 40% | 12.2 nm | 676 | cps |

### Example 2

To simulate the dissolution of human meibum with cleansing formulations of the present disclosure, castor oil was used as a model molecule. Different dissolution set-ups for castor oil in the present cleansing formulations were conducted, for example: 1) adding 0.5 gm to 5 gm of castor oil into 100 mL of a cleansing formulation in accordance with the present disclosure and mixing at body temperature (e.g. ˜37° C.) for period of time (e.g., 18 hours) with a magnetic bar and a stir mixer (Fischer Scientific, 60-700 RPM); 2) adding 0.5 gm of castor oil into 100 mL of a cleansing formulation in accordance with the present disclosure and mixing at ambient room temperature (e.g., 20-25° C.) for a period of time (e.g., a couple of months) with a slow orbital shaker (Benchmark, ˜60 RPM); and 3) adding 0.5 gm of castor oil into 100 mL of a cleansing formulation in accordance with the present disclosure placed at ambient room temperature without mixing (control).

Throughout the mixing process (e.g. 1 hour, 2 hours, 18 hours), sample from the mixture was collected for testing with a submicron particle size analyzer (PSS 380, manufactured by Particle Sizing Systems, Santa Barbara, Calif.). Particle size distribution in the nanometer range was obtained, side-by-side in comparison with that before the addition of castor oil. In addition, throughout the mixing process (e.g., 1 hour, 2 hours, 18 hours), the mixture was visually examined to observe the cloudiness and/or the completion of the dissolution, side-by-side in comparison with the control (no mixing).

Compositions in accordance with the general formulation set forth above in Table 2 were prepared containing 1%, 3%, 5%, 8%, 10%, 15%, 20%, 25%, 30%, 35%, 40%, and 50% polyoxyl 35 castor oil and used in the castor oil dissolution study. Some formulations included HEC or gellan gum as a thickener, and some formulations included aminomethylpropanol instead of tromethamine in the buffering system.

The dissolution study of castor oil with the present cleansing formulations involved several variables, such as: 1) mixing temperature; 2) mixing speed; 3) mixing time; 4) concentration of polyoxyl 35 castor oil in the formula; and 5) amount of castor oil applied.

In one aspect, the dissolution of castor oil in the cleansing formulation containing 40% polyoxyl 35 castor oil as a function of time was studied. Specifically, the 40% formulation included the following ingredients in the amounts listed below in Table 4.

TABLE 4
40% Polyoxyl 35 Castor Oil Formulation

| mg/mL | Ingredient | Percentage |
|---|---|---|
| Qs. 1.0 mL | Water for Injection, USP | Qs. 100% |
| 0 | Hydroxylethyl Cellulose, | 0% |
| | 250 M, NF | |
| 11.2 | Boric Acid, NF | 1.12% |
| 6.50 | Tromethamine | 0.65% |
| 4.00 | PEG 400, USP | 0.4% |
| 3.00 | Propylene Glycol, USP | 0.3% |
| 5.00 | Sorbitol, 70% Solution, USP | 0.35% |
| 5.00 | Polysorbate 80 | 0.5% |
| 400.00 | Polyoxyl 35 Castor Oil NF | 40% |
| 5.00 | Glycerine, USP | 0.5% |
| 100.00 | Water for Injection, USP | 10.0% |
| 2.00 | Stabilized Chlorine Dioxide, | 0.01% |
| | 5% Solution | |
| 2.00 | Polyhexamethylene Biguanide | 0.0002% |
| | (PHMB), 0.1% Solution | |

A systematic study on the foregoing cleansing formulation containing 40% polyoxyl 35 castor oil was conducted by adding 3 gm castor oil into 100 ml of the cleansing formulation. The mixing temperature was 37° C. to simulate body temperature, and the mixing speed was ˜135 rpm. the mixing time was varied from 1 hour to about 18 hours (overnight). A submicron particle size analyzer was utilized to monitor the process of dissolution. Table 5 summarizes the visual appearance and particle size changes with mixing time.

TABLE 5
Dissolution Process of Castor Oil

| Mixing Time | | Mean Particle Size |
|---|---|---|
| (hours) | Appearance | (nm) |
| 0 | Clear | 12.2 |
| 1 | Cloudy | 17871.6 |
| 2 | Cloudy | 25769.7 |
| 3 | Cloudy | 113370.0 |
| 4 | Cloudy | 140088.1 |
| 5 | Cloudy | 305092.5 |
| 6 | Cloudy | 53678.0 |
| 11 | Clear | 21.4 |
| 18 | Clear | 17.2 |

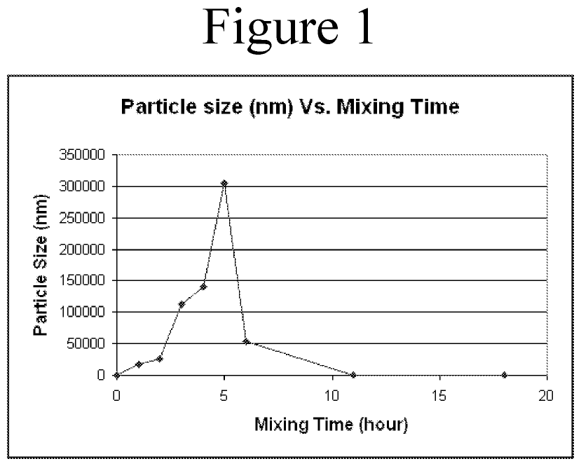

A plot of the mean particle size as a function of mixing time is presented in FIG. 1. As the mixing time increased, the mixtures with 3 gm of castor oil appeared cloudy most of the time until becoming clear after about 11 hours mixing. The particle size increased from the initial of 12.2 nm to a maximum particle size of 305 μm at 5 hours mixing, and then decreased to 21.4 nm (clear).

While not wishing to be bound by any theory, it is believed that the newly added castor oil initially formed a separate phase (oil phase) in the aqueous polyoxyl 35 castor oil formulation, and little by little as mixing continued, castor oil was absorbed by the polyoxyl 35 castor oil nanoparticles to form a new structure of larger particles. The absorbed castor oil would be expected to accumulate at the hydrophobic core area of the nanoparticles where nonpolar materials are likely to concentrate. The newly formed particles showed an increase in particle size, and such increase continued with mixing time to allow more castor oil participate in the formation of larger hydrophobic particles. The increase in particle size with mixing was observed by the submicron particle size analyzer and shown in the graph (FIG. 1). However, after all free castor oil was consumed in the polyoxyl 35 castor oil formulation, continuous mixing presumably enabled the largest hydrophobic particles of castor oil to undergo a “collapse” event in favor of smaller stable nanoparticles with less free energy. Thereafter, the overall particle size distribution demonstrated a decreasing trend with mixing time (see FIG. 1). Finally, the stable nanoparticles of about 20 nm with the lowest free energy were formed and the cloudy mixture of castor oil with the polyoxyl 35 castor oil formulation returned to a clear appearance.

A metastable particle state with maximum particle size is found in the dissolution process, which behaves as a transition state of castor oil-containing particles from kinetically energy-favored to the thermodynamically equilibrated state. The formation of such final thermodynamically stable nanoparticles with minimum free energy is believed to be rapid with mixing, correlating with the collapse events of those metastable particles with maximum particle size.

As those skilled in the art reading this disclosure will appreciate, in the practical meibum removal process, meibum materials do not necessarily need to completely dissolve in the aqueous phase for the effective removal to take place. In terms of rapidity to remove the blocking meibum material, kinetically metastable particles (whether containing castor oil or meibum materials) may be sufficient to achieve a degree of removal of a clog at the channel of a meibomian gland to provide relief to MGD patients.

Other dissolution tests of castor oil in polyoxyl 35 castor oil formulations were conducted and it was found that up to 5 gm of castor oil can be “absorbed” into 100 mL of certain polyoxyl 35 castor oil formulations, with the clear appearance occurring after a longer mixing time (e.g., 96 hours) at 37° C.

The effective removal rate of castor oil using the present cleansing formulations at the body temperature of 37° C. may be calculated by dividing the weight of total dissolved castor oil by the mixing time and by the drop size. For example, if 11 hours are needed for 3 gm castor oil to dissolve completely in 100 mL of the cleansing formulation, then:

$$RemovalRate = \frac{3 gm \times {10}^{6} μ g \text{/} gm}{11 hours \times 60 minutes \text{/} hour} \times \frac{30 μ L / drop}{100 mL \times 1000 μ L \text{/} mL} = 1.4 μ g \text{/} min / drop$$

Table 6 lists the estimated removal rates of castor oil using embodiments of cleansing formulations at the body temperature of 37° C. for several of the experiments presented above in which complete dissolution was achieved in each case.

TABLE 6

| Amount of Total | Mixing Time | | |
|---|---|---|---|
| dissolved | needed | Removal Rate | Average |
| Castor Oil (gm) | (hours) | (μg/min/drop) | Removal Rate |
| 0.5 | 3 | 0.8 | 1 μg/min/drop |
| 0.75 | 4 | 0.9 | |
| 1 | 4 | 1.3 | |
| 3 | 11 | 1.4 | |
| 4 | 40 | 0.5 | |
| 5 | <96 | >0.3 | |

Thus, in embodiments, the present formulations are capable of removing castor oil at a rate of from about 0.5 μg/min/drop to about 1.5 μg/min/drop.

### Example 3

Other systematic dissolution tests of meibum components and related materials (e.g., 0.5 gm) in compositions in accordance with the present disclosure containing 20% to 40% polyoxyl 35 castor oil (e.g., 100 mL) included the use of: 1. cholesteryl esters (cholesteryl linolenate, cholesteryl oleate, cholesteryl stearate, super sterol ester from Croda, lanolin, and lanolin oil); 2. wax esters (isopropyl palmitate, sorbitan tristearate, palmityl oleate, Jojoba oil, golden jojoba, oleyl oleate, and oleyl linoleate); 3. monoglycerides, diglycerides, triglycerides (1-oleoyl-rac-glycerol, glyceryl trioleate, coconut oil, EPA rTG, and DHA rTG); 4. phospholipids (dimyristoyl-glycero-phosphoryl-glycerol sodium or DMPG Na, lecithin (solid), lecithin (liquid), and krill oil); 5. free fatty acids (stearic acid, 12-hydroxyl stearic acid); 6. free fatty alcohol (stearyl alcohol, oleyl alcohol); 7. free sterol (cholesteryl); and 8. hydrocarbons (sequalene, mineral oil, and white petrolatum).

At the physiological temperature of 37° C., not all the studied nonpolar meibum-like materials can be “absorbed” into the compositions prepared containing 20%-40% polyoxyl 35 castor oil even with sufficient mixing, e.g. in those compositions containing mineral oil and white petrolatum. However, with heating up to >70° C., most of the nonpolar meibum-like materials demonstrated compatibility with the polyoxyl 35 castor oil formulations and clarity was maintained upon cooling.

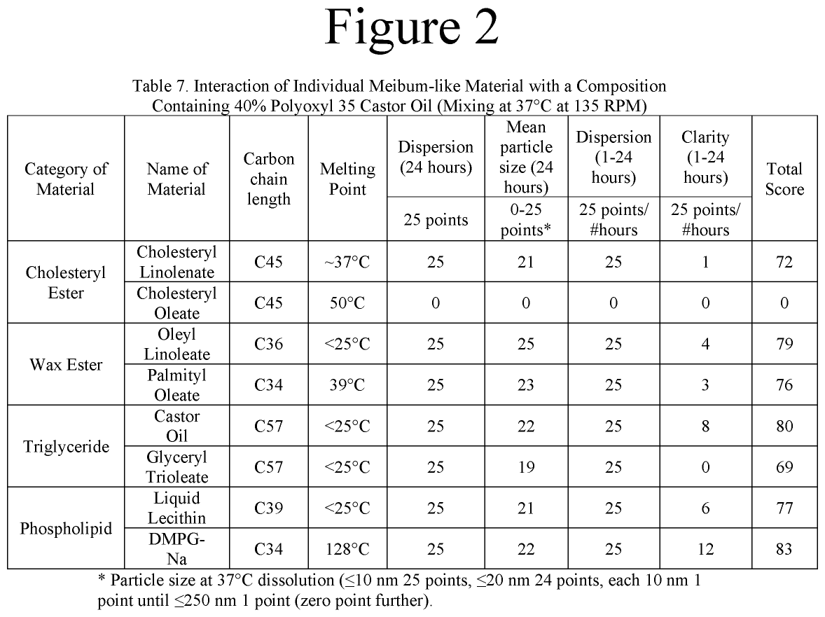

The interactions of several important meibum-like materials (0.5 gm) with a formulation containing polyoxyl 35 castor oil (100 mL) under the mixing conditions of 135 RPM at 37° C. are summarized in the Table 7 (see FIG. 2).

Those interactions in Table 7 were evaluated in terms of the homogeneity after 24 hours mixing, the mean particle size after 24 hours mixing, the minimum time to achieve homogeneous suspension, and the minimum time to achieve clarity. All of the tested articles yielded scores above 60 except cholesteryl oleate with a score of 0, which was difficult to disperse/absorb mostly due to its higher melting point. DMPG Na is a sodium salt and demonstrated the highest interaction score of 83.

Therefore, the nanoparticles present in the compositions in accordance with the present disclosure containing 40% polyoxyl 35 castor oil demonstrated the capability of absorbing nonpolar meibum materials or the like into the aqueous phase. This property can be further utilized during cleansing and removing obstructive meibum for MGD patients.

### Example 4

0.5 gm castor oil was mixed into 100 mL of formulations in accordance with the present disclosure containing different concentrations of polyoxyl 35 castor oil (e.g. see Table 2). The mixing temperature was 37° C. to simulate body temperature, the mixing speed was 700 RPM (unless the solution was too viscous to mix), and the mixing time was about 18 hours. Table 8 summarizes the results.

TABLE 8

| Concentration of | | | Particle | Particle | | |
|---|---|---|---|---|---|---|
| Polyoxyl 35 | | | Size - initial | Size - mixed | Viscosity - initial | Viscosity - mixed |
| Castor Oil | Appearance - initial | Appearance - mixed | (nm) | (nm) | (cps) | (cps) |
| 5% | Clear | Cloudy | 12.9 | 370.3 | 1.3 | n/a |
| 8% | Clear | Cloudy | 10.8 | 658.0 | 1.8 | n/a |
| 10% | Clear | Cloudy | 10.6 | 876.6 | 2.2 | n/a |
| 15% | Clear | Cloudy | 11.5 | 1786.2 | 3.8 | n/a |
| 20% | Clear | Cloudy | 12.2 | 3307.6 | 6.6 | n/a |
| 25% | Clear | Cloudy | 11.3 | 16361.1 | 18 | n/a |
| 30% | Clear | Cloudy | 10.7 | 122494.3 | 47 | 48 |
| 35% | Clear | Cloudy* | 11.7 | 23261.9 | 155 | 218 |
| 40% | Clear | Clear** | 12.2 | 32.6 | 676 | 2095 |

Before adding castor oil, the cleansing formulations “as-is” formula (initial) were all clear solutions and exhibited almost the same particle size in the range of 10 nm to 12 nm, whereas the viscosity of the formulations increased slowly with increasing concentration of polyoxyl 35 castor oil. After adding 0.5 gm castor oil into 100 mL of the cleansing formulations, the mixture appeared cloudy and kept the cloudiness until the end of the mixing (e.g. 18 hours).

To achieve a clear solution, extended mixing more than 18 hours would be required for 35% polyoxyl 35 castor oil. In fact, the mixture of 0.5 gm castor oil in 100 mL of the cleansing formula of 35% polyoxyl 35 castor oil turned out to be clear upon 60 hours continuously mixing at 37° C. For the 40% polyoxyl 35 castor oil, a clear solution was reached with complete dissolution of castor oil after 3 hours mixing at 350 RPM.

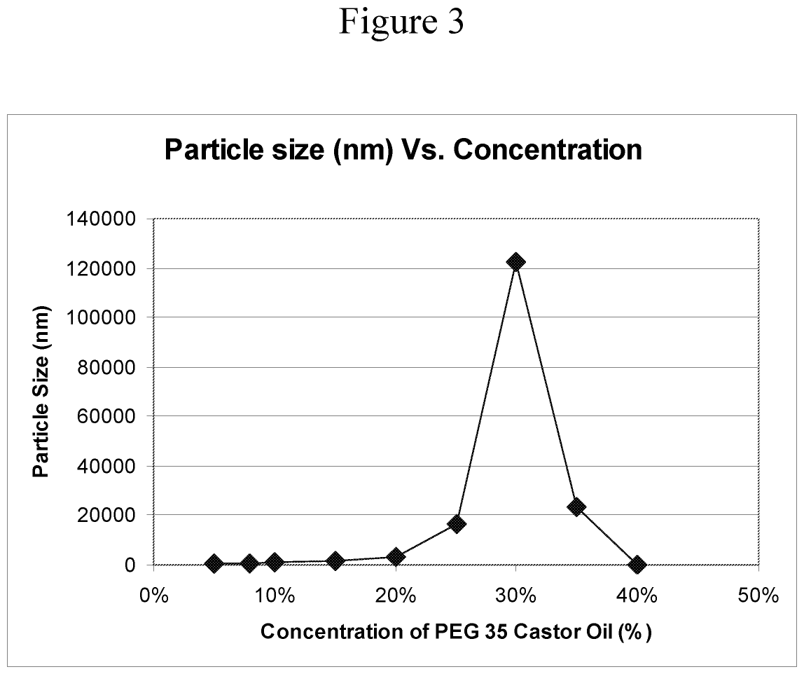

The viscosity of the mixtures increased from the initial viscosity, especially for the 40% polyoxyl 35 castor oil formula, e.g., from 676 cps to 2095 cps. The particle size increased from the initial; the observed maximum particle size (122 μm) occurred at the 30% polyoxyl 35 castor oil concentration. FIG. 3 shows a plot of the particle size change as a function of the concentration of polyoxyl 35 castor oil after 18 hours mixing for each concentration.

Interestingly, the particle size experienced an initial increase profile from 370 nm at 5% polyoxyl 35 castor oil to 122 μm at 30% polyoxyl 35 castor oil and then decreased from the maximum of 122 μm to 33 nm at 40% polyoxyl 35 castor oil. From 5%-30% polyoxyl 35 castor oil, the particle size kept increasing with the concentration. This phenomenon can be understood as the newly formed particles containing castor oil need additional mixing time to absorb the maximum amount of castor oil. For the 30% polyoxyl 35 castor oil mixture, 18 hours mixing was adequate mixing time for those castor oil containing particles to absorb the maximum amount of castor oil and therefor it reached its maximum particle size.

### Example 5

Different amounts of castor oil were added into 100 mL of a cleansing formulation in accordance with the present disclosure containing 40% polyoxyl 35 castor oil. The mixing temperature was 37° C., and the mixing speed was 60-350 RPM, depending on the mixture viscosity. The mixing time was recorded until the achievement of clear solution. Table 9 summarizes the results.

TABLE 9

| | Amount of | Mixing Time | Mixing | Particle |
|---|---|---|---|---|
| | Castor Oil | for “clear” | speed | Size |
| Experimental Condition | (gm) | solution (hours) | (RPM) | (nm) |
| 0.5 gm-5 gm of Castor | 0 (initial) | 0 | n/a | 12.2 |
| Oil → 40% Polyoxyl | 0.5 | 3 | 350 | 32.6 |
| 35 Castor Oil | 0.75 | 4 | 300 | 13.5 |
| Formulation | 1 | 4 | 300 | 10.6 |
| | 3 | 11 | 135 | 21.4 |
| | 4 | 40 | ~60 | 19.0 |
| | 5 | <96 | ~60 | 17.4 |

The formulation containing 40% polyoxyl 35 castor oil was shown to have the ability to absorb castor oil, e.g., up to 5 gm of castor oil can be dissolved completely per 100 mL of the formulation.

### Example 6

0.5 gm of castor oil was added into 100 mL of a cleansing formulation in accordance with the present disclosure containing 40% polyoxyl 35 castor oil at ambient room temperature (20° C.-25° C.) using a rather slow mixer (e.g., a Benchmark orbital shaker) with rotation speed of ˜60 rpm. It was found that about two months continuous mixing was needed to completely “dissolve” castor oil.

### Example 7

Artificial meibum was prepared by combining the ingredients shown in the following Table 10:

TABLE 10

| mg/gm | Ingredients | Percentage |
|---|---|---|
| 500.00 | Cholesterol Ester/C10-C30 | 50% |
| | Cholesterol/Lanosterol Ester | |
| 400.00 | TAG/Triglyceride/DHA | 40% |
| | rTG Fish Oil | |
| 45.00 | Freed Fatty Acid/Steraric | 4.50% |
| | Acid | |
| 50.00 | Phospholipid/DMPG-Na | 5.0% |
| 5.00 | Water | 0.5% |

A meibum dissolution study was conducted by mixing (e.g., at 250 RPM) 0.1 gm-0.5 gm of artificial meibum in 50 mL-100 mL of cleansing formulations in accordance with the present disclosure at 37° C. for 2 hours (mixing for longer time was tried but resulted in no significant difference). Side-by-side, the same amount of artificial meibum was mixed with purified water as a control. After the completion of mixing, both the meibum/cleansing formula and meibum/purified water mixtures were recovered in terms of undissolved meibum materials, either gravimetrically (by membrane filtration) or volumetrically (by sedimentation using a volumetric burette). The recovered meibum from the purified water preparation less the recovered meibum from the cleansing formula is expressed as the dissolved/absorbed meibum and calculated in percentage.

Gravimetric recovery was done in the earlier stage of the meibum study. The undissolved meibum was recovered via membrane filtration (e.g. filter through a 0.45 μm or 1 μm MILLIPORE membrane filter). The reproducibility was not satisfactory because of significant variations. Meibum is an oily, glue-like material, which can easily attach to the wall of the filter house during the recovery operation. Loss of meibum materials during membrane filtration is difficult to control considering the small sampling size of 0.1 gm-0.5 gm. Volumetric recovery was conducted by transferring the mixed meibum/aqueous suspension into a burette (e.g., 50 mL) and allowing the suspended meibum to separate out (sedimentation of meibum happens at the top of the mixture because of its lower density). A heating gun can be employed to assist the collection of the trapped meibum.

Thereafter, the volumes of the separated meibum from the meibum/cleansing formulation and the meibum/purified water mixtures were compared side-by-side and quantitated. The volume found from the cleansing formula is calculated against the volume from the control and a percentage in recovery is thereafter calculated.

The volume of recovered meibum in mL (V₁) from the cleansing formula was calculated against the volume from the purified water (V₀). The dissolved/absorbed meibum in the cleansing formula in percentage equals:

$$\% = \frac{{V}_{0} - {V}_{1}}{{V}_{0}} \times 100 \%$$

In addition, the particle size of the cleansing formula can be monitored before and after the meibum dissolution mixing, using a Submicron Particle Sizer (PSS 380, manufactured by Particle Sizing Systems, Santa Barbara, Calif.). Although the obtained particle size change cannot be quantitated to the actual amount of meibum dissolved, the increase of the particle size is generally consistent with the dissolving events of the meibum materials by the cleansing formulas.

When the meibum was mixed with the cleansing formula and purified water at 250 RPM at 37° C., the meibum was dispersed almost uniformly in the cleansing formula preparation but the meibum appeared like a large chunk/ball in the purified water preparation. Such difference can be explained as “wetting” for the cleansing formula (or the tendency to dissolve), whereas “non-wetting” for purified water (or the tendency to separate).

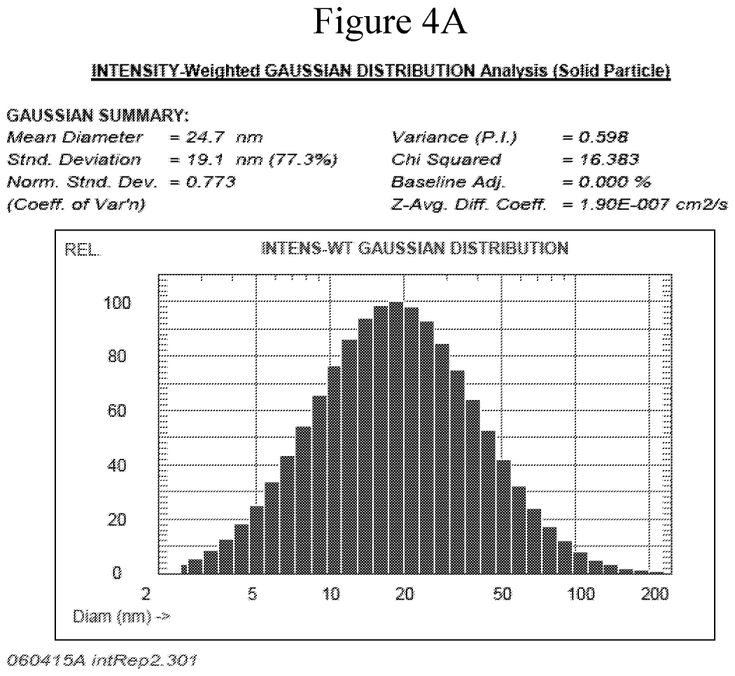

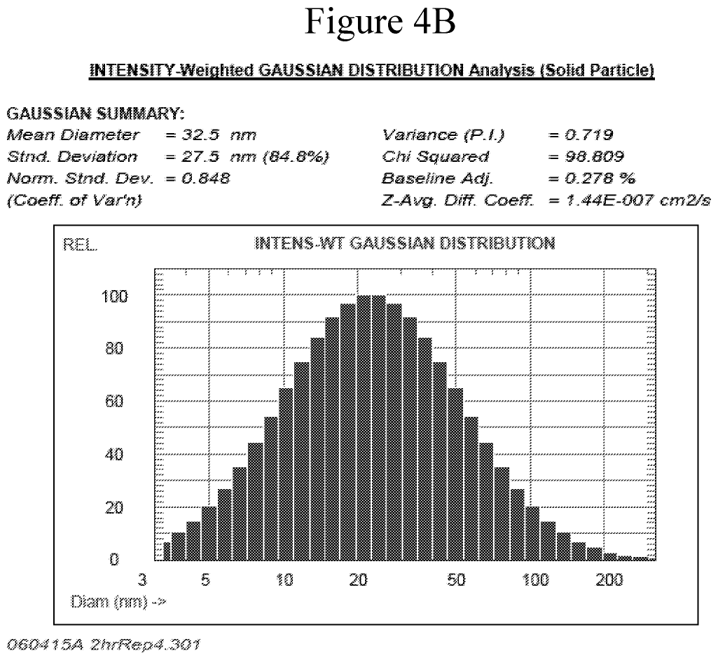

Particle size analysis for the cleansing formula was measured before and after the interaction with meibum with the Submicron Particle Sizer (Particle Sizing Systems, Santa Barbara, Calif.), as indicated in FIG. 4. Similar particle size analysis was performed on the control, but there was no particle size distribution for the purified water before the interaction.

All of the particle sizes of the cleansing formula preparations with polyoxyl 35 castor oil ranging from 5% to 10% (using the formulation vehicle shown in Table 11) were tested before and after the meibum interaction (i.e., mixed at 250 RPM, 37° C., 2 hours) with the Submicron Particle Sizer. The average particle sizes as indicated in Table 12.

TABLE 11

| mg/mL | Ingredient | Percentage |
|---|---|---|
| Qs. 1.0 mL | Water for Injection, USP | Qs. 100% |
| 1.00 | Hydroxylethyl Cellulose, | 0.1% |
| | 250 M, NF | |
| 11.2 | Boric Acid, NF | 1.12% |
| 6.50 | Tromethamine | 0.65% |
| 4.00 | PEG 400, USP | 0.4% |
| 3.00 | Propylene Glycol, USP | 0.3% |
| 5.00 | Sorbitol, 70% Solution, USP | 0.35% |
| 80.00 | Polyoxyl 35 Castor Oil NF | 8% |
| 5.00 | Glycerine, USP | 0.5% |
| 100.00 | Water for Injection, USP | 10.0% |
| 2.00 | Stabilized Chlorine Dioxide, | 0.01% |
| | 5% Solution | |
| 2.00 | Polyhexamethylene Biguanide | 0.0002% |
| | (PHMB), 0.1% Solution | |

TABLE 12
Table 3. Particle size results before and after the Meibum Interaction study

| Concentration of Polyoxyl 35 Castor oil | 5% | 6% | 7% | 8% | 9% | 10% |
|---|---|---|---|---|---|---|
| Initial (nm) | 19.8 | 21.4 | 22.5 | 25.9 | 24.7 | 20.2 |
| Final (nm) | 653.5 | 1594 | 89.5 | 31.3 | 32.5 | 26.6 |

The percentage of polyoxyl 35 castor oil varied from 5% to 50%. However, precipitation was found at ≥10% of polyoxyl 35 castor oil using the above formulation vehicle. Therefore, a few ingredients in the above formula need to be adjusted (reduced or completely taken out) when a formula with more than 10% of polyoxyl 35 castor oil was prepared.

The dissolved/absorbed meibum for the cleansing formula preparations where the polyoxyl 35 castor oil ranging from 5% to 10% (see the formula shown in Table 2) was calculated after the meibum interaction experiments (250 RPMs, 37° C., 2 hours) with the cleansing formula and purified water. Table 13 is a list of the dissolved/absorbed meibum results in percentage.

TABLE 13

| Concentration of Polyoxyl 35 Castor oil | 5% | 6% | 7% | 8% | 9% | 10% |
|---|---|---|---|---|---|---|
| Absorbed (%) | 23% | 26% | 31% | 40% | 26% | 29% |

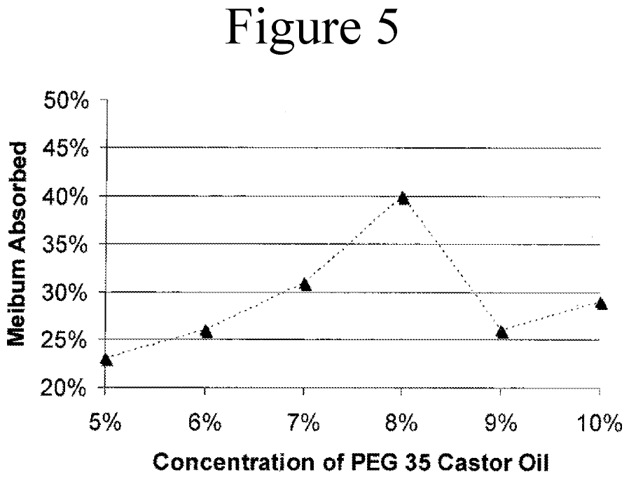

To further illustrate the absorption results, FIG. 5 shows a plot of the meibum dissolved/absorbed in percentage as a function of the concentration of polyoxyl 35 castor oil (5%-10%). The maximum meibum dissolved/absorbed in percentage (˜40%) has been found to correspond to ˜8% polyoxyl 35 castor oil in the cleansing formula.

### Example 8

In order to further explore the capability of meibum absorption by the nanoparticles in formulations containing polyoxyl 35 castor oil, the buffering agents (boric acid and tromethamine) and thickener (e.g., HEC) were removed from the cleansing formulation allowing the concentration of polyoxyl 35 castor oil to be increased to 50%. The formula for the 50% formulation is shown in Table 14.

TABLE 14

| mg/mL | Ingredient | Percentage |
|---|---|---|
| Qs. 1.0 mL | Water for Injection, USP | Qs. 100% |
| 4.00 | PEG 400, USP | 0.4% |
| 3.00 | Propylene Glycol, USP | 0.3% |
| 5.00 | Sorbitol, 70% Solution, USP | 0.35% |
| 500.00 | Polyoxyl 35 Castor Oil NF | 50% |
| 5.00 | Glycerine, USP | 0.5% |
| 100.00 | Water for Injection, USP | 10.0% |
| 2.00 | Stabilized Chlorine Dioxide, 5% Solution | 0.001% |
| 2.00 | PHMB, 0.1% Solution | 0.0002% |

The appearance of the 50% polyoxyl 35 castor oil formulation is a clear liquid gel at room temperature. The regular meibum dissolution study cannot be performed due to its high viscosity. However, 25% polyoxyl 35 castor oil using the formulation base in Table 14 was prepared and appears as a clear solution. A meibum dissolution study on 25% cleansing formula resulted in about 50% meibum absorption. This improved meibum absorption result is consistent with a conclusion that greater numbers of nanoparticles resulting from greater concentrations of polyoxyl 35 castor oil means more polarity “vacancies” available for absorption and leads to enhanced meibum absorption.

When the percentage of polyoxyl 35 castor oil is 50%, the resulting preparation demonstrated the characteristics of a transparent liquid gel. Such gel can be utilized as ophthalmic insert, which can be administered inside the eyelid and/or as eyelid wipes to cleanse the entire surface of the eyelids.

Chemically, there is a degree of similarity of the molecular structure of polyoxyl 35 castor oil and wax/cholesterol ester molecules such as those found in meibum (e.g., both molecules share the similar glycerol backbone structure as the hydrophilic side) and the similar length of the fatty acid chains (e.g., both have long chains of such as eighteen carbon atoms as the hydrophobic side) can give rise to an affinity of the meibum materials (wax esters/cholesterol esters/triglycerides) towards polyoxyl 35 castor oil in the presently described cleansing formulations, and therefore lead to dissolution and/or dispersion of those meibum materials. The hydrophilic-hydrophobic polarity and the structure similarity of polyoxyl 35 castor oil can initiate the solubility/affinity of the meibum materials in the self-assembled nanolipids contained in the presently described cleansing solutions. The dissolving/dispersing events of meibum materials in the cleansing formulations is also shown by increases in the average particle size for the nanolipids of the cleansing formulations. Some of the meibum materials were believed to be stabilized (absorbed) via the hydrophobic “core” of nanolipids, manifesting the overall size increase.

The coexistence of polarity and chemical structure similarity of polyoxyl 35 castor oil molecules makes it efficient in dissolving/absorbing the meibum materials using the presently described cleansing formulations. When the concentration of polyoxyl 35 castor oil varied from 5% to 10% in one study, the dissolved/absorbed meibum material was found to increase from 23% to 40%, whereas the maximum meibum absorption (40%) occurs at 8% of polyoxyl 35 castor oil in the cleansing formulations tested. In general, increasing the concentration of polyoxyl 35 castor oil, and hence increasing the population of nanolipids/nanoparticles, provides more polarity “vacancies” leading to more of the meibum materials being absorbed.

### Example 9

The safety of microemulsion compositions in accordance with embodiments of the present disclosure containing, e.g., 5%-40% of polyoxyl 35 castor oil was evaluated via Draize test and MTT assay. Microemulsion compositions in accordance with embodiments of the present disclosure with 8%, 15%, 25%, and 50% of polyoxyl 35 castor oil polyoxyl 35 castor oil were used in an ocular irritation study (e.g., a Draize rabbit eye test). The study design and results are summarized in Table 15.

TABLE 15
Ocular Irritation Tests

| Animal | | | | |
|---|---|---|---|---|
| Number (n) | Treatment | Eyes | Dose (mL) | Dose schedule |
| 3 New Zealand | Test | Right (Test) | 0.1 | Once a Day |
| White Rabbits | Control | Left (Control) | 0.1 | Once a Day |

| Concentration of | | | |
|---|---|---|---|
| polyoxyl 35 castor oil | Dose schedule | Scoring | Results* |
| 8% | Once 0.1 mL | 1 hr ± 6 min | Not positive |
| | | 24 ± 2 hrs | irritation response |
| 15% | Once 0.1 mL | 48 ± 2 hrs | Not positive |
| | | 72 ± 2 hrs | irritation response |
| 25% | Once 0.1 mL | | Not positive |
| | | | irritation response |
| 50% | Once 0.1 mL | | Not positive |
| | | | irritation response |

The Draize test was conducted by applying the stated dosage of the microemulsion composition on three individual rabbits and observing for any indication of irritation. Scores were recorded at 1 hour, 24 hours, 48 hours, and 72 hours after dosing. No positive irritation response was found for any of the studied compositions.

A microemulsion preparation in accordance with an embodiment of the present disclosure with 40% polyoxyl 35 castor oil was used for an ocular irritation study (a Draize rabbit eye test with an additional challenge). The study design and the results are summarized in Table 16.

TABLE 16
Challenged Ocular Irritation Tests

| Animal | | | | |
|---|---|---|---|---|
| Number (n) | Treatment | Eyes | Dose | Dose schedule |
| 3 New Zealand | Test | Right (Test) | 60 μL | Twice a Day |
| White Rabbits | | | | (approximately 6 to |
| | | | | 8 hours apart) for |
| | | | | 10 consecutive days |
| | Control | Left (Control) | 60 μL | Twice a Day |
| | | | | (approximately 6 to |
| | | | | 8 hours apart) for |
| | | | | 10 consecutive days |

| Concentration of | | | |
|---|---|---|---|
| Polyoxyl 35 Castor oil | Dose schedule | Scoring | Results* |
| 40% | Day 1, two | 1 hr ± 6 min | No positive |
| | drops, twice | post dosing | irritation response |
| | Day 2, two | Prior to next | No positive |
| | drops, twice | dosing | irritation response |
| | Day 3, two | | No positive |
| | drops, twice | | irritation response |
| | Day 4, two | | No positive |
| | drops, twice | | irritation response |
| | Day 5, two | | No positive |
| | drops, twice | | irritation response |
| | Day 6, two | | No positive |
| | drops, twice | | irritation response |
| | Day 7, two | | No positive |
| | drops, twice | | irritation response |
| | Day 8, two | | No positive |
| | drops, twice | | irritation response |
| | Day 9, two | | No positive |
| | drops, twice | | irritation response |
| | Day 10, two | | No positive |
| | drops, twice | | irritation response |

The Draize test was conducted by performing the repeated dosage of the microemulsion containing 40% polyoxyl 35 castor oil in accordance with an embodiment of the present disclosure on three individual rabbits for ten (10) consecutive days, and observing for any indication of irritation. Scores were recorded at each study day prior to next dosing. No positive irritation response was found for the microemulsion containing 40% polyoxyl 35 castor oil.

Another ocular irritation study was done by exposing a microemulsion containing polyoxyl 35 castor oil in accordance with an embodiment of the present disclosure onto normal human keratinocytes/stratified squamous epithelium which is similar to that found in the cornea epithelium of the human eye (EPIOCULAR model), and then checking tissue viability with MTT assay for cytotoxicity after incubation in comparison with a water control (Cyprotex). The study design and results are summarized in Table 17.

TABLE 17
Ocular Irritation Tests - MTT Assays

| | Mean Tissue* | Irritation Response |
|---|---|---|
| Sample ID | Viability | (Irritant if ≤ 60%) |
| Methyl Acetate | 31.1% | Positive |
| (Positive Control) | | |
| Water | 100.0% | Negative |
| (Negative Control) | | |

Concentration of Polyoxyl 35 Castor oil

| | | |
|---|---|---|
| 25% microemulsion | 98.4% | Not Irritant |
| 40% microemulsion | 102.4% | Not Irritant |

As the foregoing data shows, microemulsion formulations containing 5%-40% polyoxyl 35 castor oil in accordance with embodiments of the present disclosure demonstrated “nonirritant response” in the ocular irritation (OIT) studies, including both Draize test and the MTT assay for cytotoxicity. Microemulsion formulations containing 40% polyoxyl 35 castor oil in accordance with embodiments of the present disclosure ware further challenged with a customized Draize test under repeated dosage conditions for up to ten (10) days. Those studies consistently suggest that the present compositions with 5%-40% polyoxyl 35 castor oil were not irritating towards the New Zealand White rabbit's eyes in Draize test and/or the tissues/normal human keratinocytes utilized in the MTT assay for cytotoxicity. Accordingly, microemulsions in accordance with embodiments of the present disclosure containing polyoxyl 35 castor oil in the range of 5%-40% are shown to be safe to use as ophthalmic preparations and safe to use for the purpose of cleansing the eyes.

In summary, the presently described cleansing formulations provide a chemical eye drop treatment for MGD, targeting the cleansing of obstructive meibum materials that may be positioned in the orifice of meibomian gland channels. To conduct a meibum dissolution study, artificial meibum was prepared to generally reflect the composition of human meibum. Formulations in accordance with the present disclosure were prepared with various concentrations of polyoxyl 35 castor oil, corresponding to different amounts of nanoparticles. The meibum materials demonstrated “wetting” in the present cleansing formulations and the dissolved/absorbed meibum increases in percentage with increasing concentration of polyoxyl 35 castor oil (e.g., from 5% to 8%). The particle size of the present cleansing formulations was found to increase upon exposure to the meibum materials, consistent with the absorption of the meibum materials in the hydrophobic “core” of the nanoparticles. The dissolved/absorbed meibum was found to be 40% for a tested cleansing formula with 8% polyoxyl 35 castor oil, and to be 50% for a tested cleansing formula with 25% polyoxyl 35 castor oil. While not wishing to be bound by any theory, the interaction of nanoparticles towards the meibum materials appears to be based on the similar hydrophilic-hydrophobic polarity of polyoxyl 35 castor oil and the chemical structural similarity of the polyoxyl 35 castor oil molecule and the meibum materials. A toxicity/ocular irritation study on cleansing formulations in accordance with the present disclosure having the highest concentration of polyoxyl 35 castor oil (50%) with the New Zealand white rabbits demonstrated the present formulations were not irritating.

Various modifications of the disclosed concepts and embodiments, in addition to those described herein, will be apparent to those skilled in the art from the foregoing description. Various modifications, substitutions, and variations can be made to the disclosed embodiments without departing from the essential characteristics of the present disclosure. As such, any modifications, variations, or substitutions, which may occur to those skills in the art, should be considered to be within the scope of the present disclosure.

### REFERENCES

The following publications are incorporated herein by references in their entirety:
1. Cremophor® EL Castor Oil, Technical Information, June 2008, published by BASF.
2. Osgood J. K., Dougherty J. M., and McCulley J. P., The Role of Wax and Sterol Esters of Meibomian Secretions in Chronic Blepharitis, Inv. Ophthal. & Vis. Sci., 30 (1989) 1958-1961.
3. Patel, S., Nelson, D. R., and Gibbs, A. G., Chemical and physical analyses of wax esters properties, J. Ins. Sci., 1.4. (2001).
4. Nelson J. D., Shimazaki J., Benitez-del-Castillo J. M., Craig J. P., McCulley J. P., Den S., and Foulks G. N., The International Workshop on Meibomian Gland Dysfunction: Report of the Definition and Classification Subcommittee, Ophthal. & Vis. Sci., 52 (2011) 1930-1937.
5. Butovich I. A., The Meibomian Puzzle: Combining Pieces Together, Prog Retin Eye Res. 28 (2009) 483-498.
6. Butovich I. A., Human tear film and meibum. Very long chain wax esters and (O-acyl)-omega-hydroxy fatty acids of meibum, J. Lipid Res. 50 (2009) 2471-2485.
7. Butovich I. A., Cholesteryl esters as a depot for very long chain fatty acids in human meibum, J. Lipid Res. 50 (2009) 501-513.
8. Butovich I. A., Fatty acid composition of cholesteryl esters of human meibomian gland secretions, Steroids, 75 (2010) 726-733.
9. Butovich I. A., Arciniega, J. C., Lu, H., and Molai, M., Evaluation and quantitation of intact wax esters of human meibum by gas-liquid chromatography-ion trap mass spectrometry, IOVS, 53 (2012) 3766-3781.
10. Shrestha, R. K., Borchman, D., Foulks, G. N., Yappert, M. C., and Milliner, S. E., Analysis of the composition of lipid in human meibum from normal infants, children, adolescents, adults, and adults with meibobian gland dysfunction using ¹H-NMR spectroscopy, IOVS, 52 (2011) 7350-7356.
11. Brown S. H. J., Kunnen, C. M. E., Duchoslav, E., Dolla, N. K., Kelso, M. J., Papas, E. B., de la Jara, P. L., Willcox, M. D. P., Blanksby, S. J., and Mitchell, T. W., A Comparison of Patient Matched Meibum and Tear Lipidomes, Inv. Ophthal. & Vis. Sci., 54 (2013) 7417-7423.
12. Kunnen, C. M. E., Brown, S. H. J., de la Jara, P. L., Holden, B. A., Blanksby, S. J., Mitchell, T. W., and Papas, E. B, Influence of Meibomian Gland Expression Methods on Human Lipid Analysis Results, Ocul. Surf, 14 (2016) 49-54.
13. Pucker, A. D. and Nichols, J. J., Analysis of Meibum and Tear Lipids, Ocul. Surf, 10 (2012) 230-250.
14. Rantamäki, A. H., Seppänen-Laakso, T., Oresic, M., Jauhiainen M., Holopainen, J. M., Human Tear Fluid Lipidome: From Composition to Function, PloS One, 6-5 (2011) e19553.
15. Lam, S. M., Tong, L., Yong, S. S., Li, B., Chaurasia, S. S., Shui, G., and Wenk, M. R., Meibum Lipid Composition in Asians with Dry Eye Disease, PloS One, 6-10 (2011) e24339.
16. Mori, N. Fukano, Y., Arita, R., Shirakawa, R., Kawazu, K., Nakamura, M., and Amano, S., Rapid identification of fatty acids and (O-acyl)-w-hydroxy fatty acids in human meibum by liquid chromatography/high-resolution mass spectrometry, J. Chrom. A, 1347 (2014) 129-136
17. Hardwick S. J., Carpenter, K. L., Law, N. S., van der Veen, C., Marchant, C. E., Hird, R., and Mitchinson, M. J., Toxicity of polyunsaturated fatty acid esters for human monocyte-macrophages: the anomalous behavior of cholesteryl linolenate, Free Radic. Res., 26 (1997) 351-363.
18. Shine W. E. and McCulley J. P., Role of Wax Ester Fatty Alcohols in Chronic Blepharitis, Ophthal. & Vis. Sci., 34 (1993) 3515-3521.
19. Nichols K. K., Ham B. M., Nichols J. J, Ziegler C., and Green-Church K. B., Identification of Fatty Acids and Fatty Acid Amides in Human Meibomian Gland Secretions, Ophthal. & Vis. Sci., 48 (2007) 34-39.
20. Dalton J. T. and Eswaraka J., Methods of Treating Meibomian Gland Dysfunction, U.S. Pat. No. 8,791,158 B2 (2014).
21. Goto E., Shimazaki J., Monden Y., Takano Y., Yagi Y., Shimmura S., and Tsubota K., Low-concentration Homogenized Castor Oil Eye Drops for Noninflamed Obstructive Meibomian Gland Dysfunction, Ophthalmology, 109 (2002) 2030-2035.
22. Tah V., Saha K., Myerscough J., Ahad M., Ho J., Sharma P., Tariq F., and Tuft S., Dry Eye—An Insight into Meibomian Gland Dysfunction, Ophthalmology—Current Clinical and Research Updates, Chapter 7 (2014).
23. Craig J. P., Purslow C., Murphy P. J., and Wolffsohn J. S. W., Effect of a liposomal spray on the pre-ocular tear film, Cont Lens Anterior Eye, 33 (2010) 83-87.
24. Zhao, L., Chen, X. J., Zhu, J. et al., Lanosterol reverses protein aggregation in cataracts, Nature, 523 (2015) 607-611.
25. Qiao J. and Yan, X., Emerging treatment options for meibomian gland dysfunction, Clin. Ophthal. 7 (2013) 1797-1803.
26. Sawaya, A. S., Composition for a topical ophthalmic clear colloid liquid which undergoes a liquid-gel phase transition in the eye, U.S. Pat. No. 9,006,214 B2 (April 2015).
27. Xia, E. and Fridman, K., Ophthalmic Composition with Alkoxylated Natural Waxes, U.S. Pat. No. 9,375,401 B2 (June 2016).
28. Coffey M. J., Ophthalmic Composition with Wax Esters, US Patent 2015/0202306 A1 (July 2015).
29. Dalton, J. T. and Eswaraka, J., Methods of Treating Meibomian Gland Dysfunction, U.S. Pat. No. 8,791,158 B2 (July 2014).
30. Millar, T. and Schuett, B., Ophthalmic Formulation, US Patent 0377335 A1 (December 2014)
31. Maskin S., Treatment for Meibomian Gland Dysfunction or Obstruction, U.S. Pat. No. 8,906,427 B2 (2014).

What is claimed is:

1. An eye treatment formulation comprising;
nanoparticles formed by at least one polyoxyethylene castor oil derivative, the nanoparticles being present in an amount sufficient to absorb meibum material, and
wherein the formulation exhibits a clear appearance.

2. The eye treatment formulation of claim 1 wherein the nanoparticles have a mean size of from about 5 nm to about 100 nm prior to absorbing meibum material.

3. The eye treatment formulation of claim 1 wherein the nanoparticles are present in an amount sufficient to absorb at least one cholesteryl ester or at least one wax ester.

4. The eye treatment formulation of claim 1 wherein the at least one polyoxyethylene castor oil derivative includes polyoxyl 35 castor oil.

5. The eye treatment formulation of claim 4 wherein polyoxyl 35 castor oil is present in the formulation at a concentration of from about 1% to about 50%.

6. The eye treatment formulation of claim 4 wherein polyoxyl 35 castor oil is present in the formulation at a concentration of from about 8% to about 40%.

7. The eye treatment formulation of claim 4 wherein polyoxyl 35 castor oil is present in the formulation at a concentration of from about 18% to about 35%.

8. The eye treatment formulation of claim 1 further comprising one or more of a buffer, a lubricant/demulcent agent, or a preservative.

9. A method comprising:
administering an eye treatment formulation to a patient experiencing a dry eye condition resulting from meibomian gland dysfunction, the eye treatment formulation including nanoparticles formed by at least one polyoxyethylene castor oil derivative, the nanoparticles being present in an amount sufficient to absorb meibum material, and wherein the eye treatment formulation exhibiting a clear appearance.
